(19) United States
(12) Patent Application Publication
(10) Pub. No.: US 2007/0243227 A1
(43) Pub. Date: Oct. 18, 2007

(54) Coatings for surgical staplers

(76) Inventor: Michael Gertner, Menlo Park, CA (US)

Correspondence Address: MICHAEL GERTNER, P.O. BOX P, MENLO PARK, CA, 94026, US

(21) Appl. No.: 11/786,897

(22) Filed: Apr. 13, 2007

Related U.S. Application Data
(60) Provisional application No. 60/744,913, filed on Apr. 14, 2006.

(51) Int. Cl. A61F 2/00

(52) U.S. Cl. 424/424

(57) ABSTRACT

A material with a first state and a second state is applied to a surgical stapler. The stapler is applied to tissue in a surgical field while the material is in the first state. Subsequently, the second state is achieved by the material to secure the staple line. In some embodiments, bioactive agents are eluted from or into the staple line.

### BACKGROUND

Surgical staplers 70 (FIG. 1) are devices which have been used for several decades. Generally and irrespective of the tissue or type of device, the mechanism is the same. Staples are pushed out from the staple ejecting side of the stapler 50, through the tissue to be stapled together, and subsequently against the anvil side of the stapler 60 to form a B shape 30 (FIG. 1e) within the tissue, after which the tissue is pushed together within the B 30 by the staples.

The rows of the B 30 shaped staples then hold the tissue together to facilitate healing of the tissue within the B shape. Typically in the prior art, the amount of pressure applied by each staple B is not uniform, as some Bs have a good grip on the tissue and others do not have an adequate grip because there is so much compliance in the system. For example, the anvil 60 jaw often pulls away from the staple applicator claw while staples are being ejected from the stapler and pushed through the tissue during staple ejection from the staple ejection side 50.

Modern staplers also require surgeons to choose the size of the staple that ultimately becomes a B 30. There are typically 3-4 sizes of staples with correspondingly 3-4 final staple sizes after they form the B 30. The B size is determined by the thickness of the bower after stapling. However, it is difficult to determine this post-stapling thickness of the tissue prior to release of the staples; therefore, the process of choosing the B thickness is an educated guess at best.

Recently, the concept of buttressing has become important in facilitating the healing process and preventing tissue dehiscence; buttresses can make up for the lack of consistent staple height and variance of force applied by the jaws across the staple line. Staple lines are “buttressed” when a separate material is interposed within the B 30 of the staple line. The buttress performs two functions: 1) linking the staples together; 2) filling in the gap between the top of the staple and the tissue and the B portion of the staple and the tissue. With the buttress, uniformity along the length of the staple line is not as important as without the buttress.

Buttresses 75 are applied to the staplers 70 (FIG. 1F) at the time of surgery, being placed around each stapler jaw by the surgeon or the surgical technician; when the tissue is placed between the claws and compressed together, the buttress is also compressed, awaiting the staples to be pushed through the material. The result is a material interposed underneath B of the staples which fills in and makes up for the discrepancy among the Bs 30.

As can be seen in FIG. 1F, the buttresses can be tedious to apply because they do not fit snugly on the jaws and operating room staff needs to place the buttresses 75 on the stapler and then the surgeon needs to place the stapler and buttress through the laparoscopic port and into the abdomen. The buttress 75 is often bulky on the stapler 70 and difficult to fit through the laparoscopic ports for placement around the tissue. In addition, valuable operating room time is spent on the application of the buttresses to the jaws rather than the operation.

Aside from the tedium of applying the buttresses to the staplers, current buttress materials are fabrics of various sorts which add bulk but do not necessarily plug the gap of each staple. The staples can also pull away or through the fabric.

### SUMMARY OF INVENTION

In accordance with the above deficiencies of both staplers and buttress materials, the current invention describes a coating material which in one embodiment is placed onto the jaws of the stapler. The coating has two states, the first state being utilized prior to release (firing) of the staples from the stapler and the second state being utilized subsequent to firing of the staples. After the staplers are fired, the coating material can in one embodiment absorb water from the tissues. The coating material then expands into a bona-fide buttress (second state). The stapling of the coating in its first state can also cut out the buttress from the material covering the stapler jaw so that the shape of the buttress material approximately conforms to the shape of the stapler jaw.

### PRIORITY DATA

This non-provisional patent application claims priority to provisional patent application Ser. No. 60/744,913 filed Apr. 14, 2006.

### DESCRIPTION OF FIGURES

FIGS. 1a-1f depict surgical staplers and buttresses in the prior art.

FIGS. 2a-2b depict staples within the two state material.

FIGS. 3a-3b depict the material after it is applied to tissue.

FIGS. 4a-4b depict additional views of the material after it is stapled to tissue.

FIG. 5 depicts the pathway for a stapler coating to reach a human patient.

### DESCRIPTION OF INVENTION

The current invention addresses the following needs: 1) the need for a buttress that is applied by the surgeon with minimal disruption of work flow during surgery; 2) the need to fill or plug the gaps between and around staples; and 3) the need to eliminate the need to choose (i.e. guess) which final staple height is optimal for the tissue.

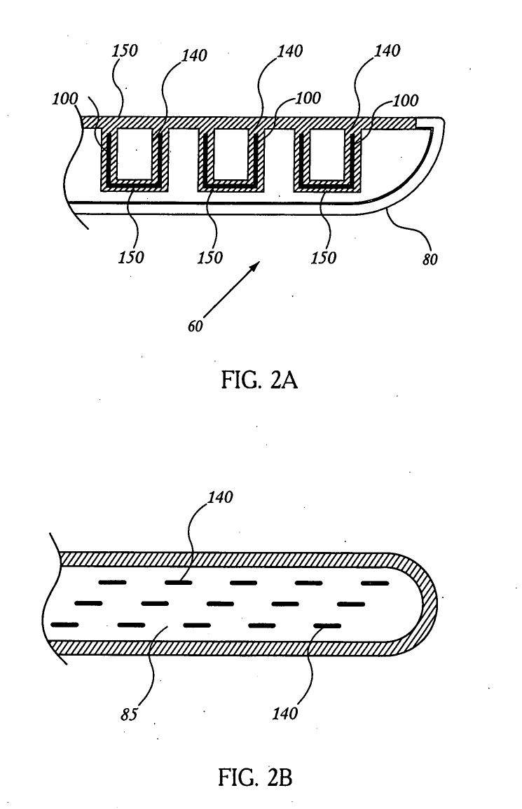

In order to achieve these goals in this invention, a two-state material 150 (FIG. 2a) is applied to the jaws of the stapler; in one state, the material can have a thinner profile than the second state. The second state is achieved after the staples are released from the stapler. One key feature of this material is that it is flexible when it reaches the second state. In some embodiments, the material is also adherent to the stapler jaws after it reaches the second state. The material 150 can also infiltrate into the holes 140 which house the staples 100 (FIG. 2A-B).

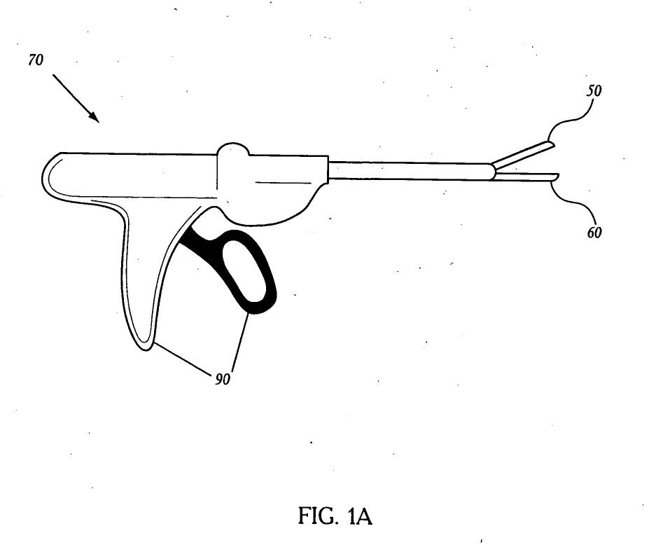

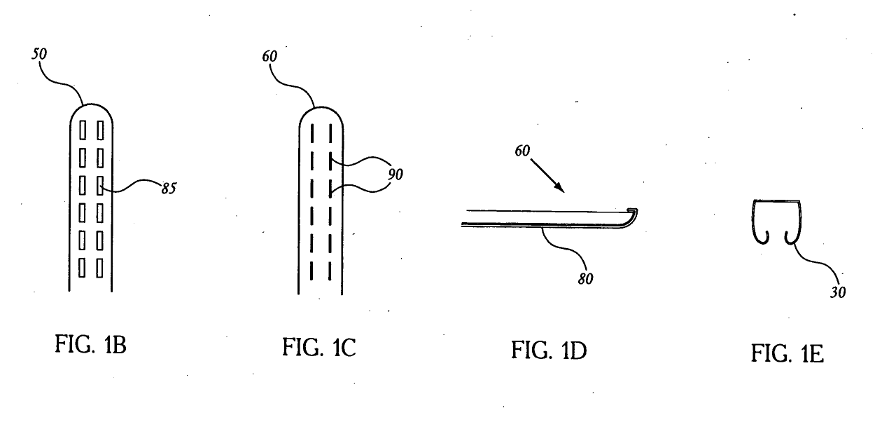

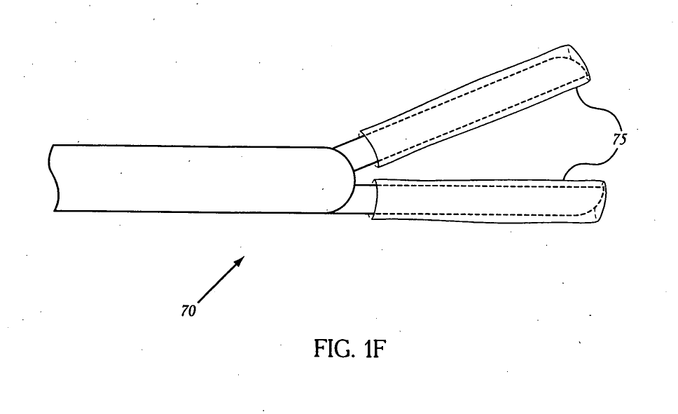

In some embodiments, the material is applied to both jaws 50 and 60 (FIG. 1); in other embodiments, the material is applied to one of the jaws 50 or 60. In some embodiments, the material is applied to substantially the entirety of the jaw and in other embodiments, the material is applied substantially only partially to the jaws. In some embodiments, the material is applied at the time of surgery; in other embodiments, the material is applied to the staple jaws prior to the time of surgery.

In one embodiment, a water based material is applied to the stapler jaws 80 (FIG. 2a) and the material 150 is allowed to dry such that the water evaporates out of the material, leaving a thin coating on the stapler jaw 80 or jaws (state 1). Staples 100 reside in their wells 140. The coating 150 can be invisible or visible to the user of the stapler at this point. The stapler is sterilized after the application of the coating and delivered to the physician. The stapler is taken out of a sterile package at surgery and then fired through the material and across the tissue.

As the staples are applied through the material 150, a buttress cutout 85 (FIG. 2b) of material is applied to the tissue at the same time the staples are applied to the tissues. In the embodiment where the anvil portion of the stapler has the two phase material applied, the staples “grab” a cutout from the anvil portion of the stapler as well, creating a continuous material across the staple line and therefore across the tissue. This step therefore avoids the need for the surgeon or surgical team to separately apply the buttress to the stapler prior to use.

Over time, water is absorbed from the tissue into the material; the material can then expand to fill the gaps in the staple line. In addition, as the material swells and pushes against the staple lines, the tissues are compressed toward one another. Because the material can expand to fill the gaps in the staple lines, it may not be necessary to choose discreet staple sizes; when the material expands, it fills the spaces left by staples that are too big relative to the amount of compressed tissue. This feature therefore allows the surgeon to choose any size staple because the material fills in the extra space between the tissue and the staple.

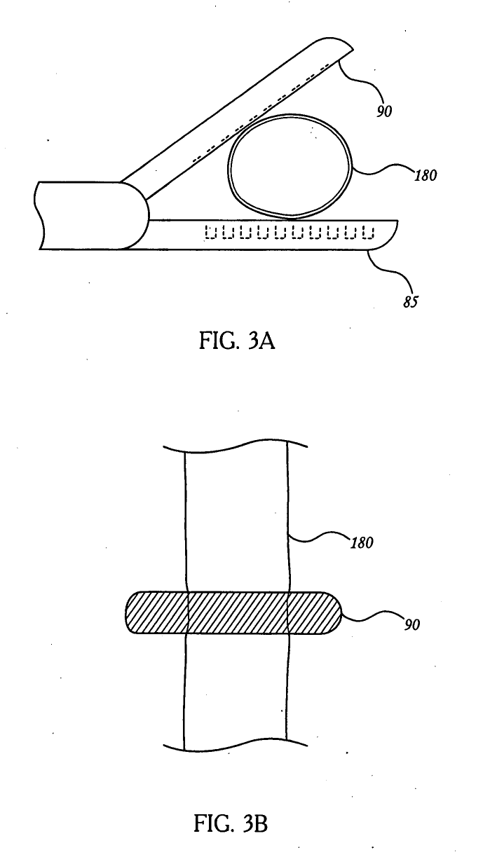

FIG. 3a depicts tissue 180 between a stapler applicator 90 and its anvil 85. FIG. 3b is a view from the top, showing the staple applicator 90 closed over tissue 180.

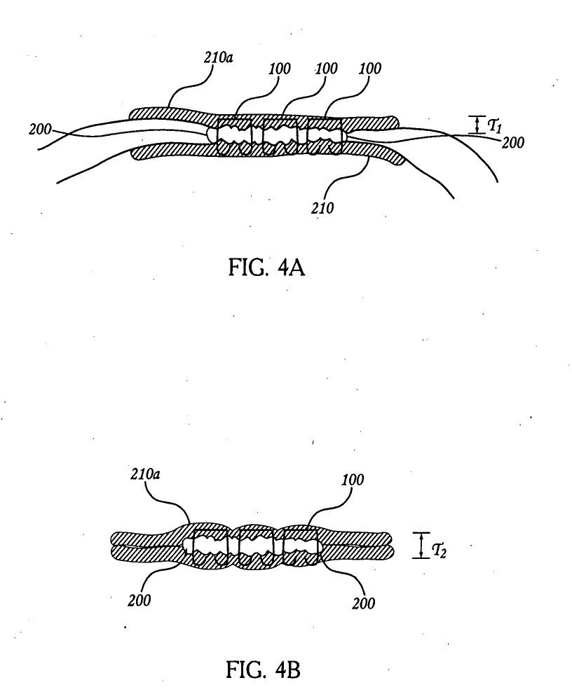

FIG. 4 depicts a cross section of a tissue with a lumen 200 after the stapler is applied and staples 100 compress the crush the luminous tissue 200 together. Material 210a is the cutout 85 depicted in FIG. 2b. Depending on the material 210a, cutout 85 may or may not overhang the region of the bowel and staples 100, as the cutout will “grab” less of the material. Material 210a is shown with thickness T1 which is the thickness just after application of the staples 100. In FIG. 4b, material 210a is shown at thickness T2 which, depending on the material 210, is the thickness of the material after water is pulled into the material 210 and the material subsequently expands thickness T2.

In some embodiments, the material 210 is a hydrogel which absorbs water to reach an equilibrium. In other embodiments, material 210 is a hydrocolloid which tends to draw in less water; however, less water leaves the material after the initial phase of coating the material; therefore, the hydrocolloid can be stronger than a hydrogel in some embodiments. The degree of cross-linking within the material 210 can determine the tensile strength between the staples which ultimately determines the strength of the anastomosis. The degree of cross-linking can also determine the amount of swelling in the case where the material is in fact a hydrophilic material such as a hydrogel.

Examples of hydrogels include polyethylene oxide (PEO), polyethylene glycol (PEG), silicone, polyacrylamides, polyethylene oxide, polyvinylpyrrolidone, polyvinyl alcohol, sodium polyacrylate, polyethylene glycol (PEG), etc.

In some embodiments, hydrogel materials are coated onto other biocompatible materials such as polypropylene, polyester, silicone, cadaveric dermis, xenogenic dermis. The composite material can then serve as a buttress. In another example, Seamgard™ produced by Gore™ Inc. can be coated with expandable materials to improve the properties of the buttresses. Similarly, PeriStrips™ from Synovis™ surgical also can be used coated in order to serve a similar purpose.

Coating materials 210 can include polymers, metals, and can include hydrogels, nanoparticles, hydrocolloids, nanotubes, nanotubes, silicon nanowires, buckyballs, metallic nanoparticles, ceramic nanoparticles, nanoparticles with coatings, cross-linked and/or polymerized nanoparticles, polyethylene glycol, agarose, and silica. Any of these materials can be mixed or matched as desired. They can be processed in a number of way including heating, drying, vacuum processing, etc. The material can be applied to the jaws of the stapler through a curing process with or without light, an evaporation process, a solvation process, a spray coating process, or a dip coating process.

Other coating materials include silicone, polyurethane, polylactides, polyanhydrides, acrylics, acrylates, or epoxies. Elastomers, either biodegradeable or non-biodegradeable, can be used as the coating for the staplers. The elastomers can be fillable or non-fillable. In one embodiment, the elastomer is applied to the stapler. The elastomer is stretched at the time the tissue is placed inside the stapler jaws and the stretched state is the state 1.

After the staples are released from the stapler and penetrate the elastomer, and the jaws are released from the tissues, the elastomer returns to its original, non-stretched state, which is considered state 2. The return of the elastomer to its first state will fill in the extra space between the staples and the tissue mitigating the problems outlined above.

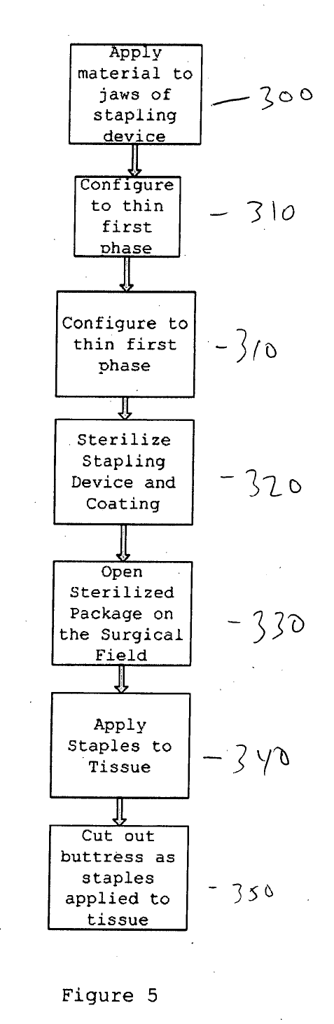

A process to apply the material coating to the surgeon is also described (FIG. 5):
1) Applying a material 300 to the stapler.
2) Forming the first phase of the material on the jaw of the stapler. The second phase can be induced by drying, heating, evaporating, curing light (e.g. UV, blue, infrared), etc. The second phase allows for easy handing and seemless integration into current surgical practice 310. Indeed in some embodiments, the material is invisible to the physician and operating room personnel.
3) The stapler is then sterilized, packaged, and sent to the physician 320.
4) The stapler is opened in the surgical field and applied to the tissue of interest 330.
5) The material is now in place above the tissue and inside the staple holes. The material is also crosslinked at this juncture so it can act as a buttress when it is peeled off the jaw of the staple.
6) Next, the staples are applied to the tissues, the staples pulling the cross-linked material off the staple jaws. As the buttress lifts off the stapler jaw, the buttress is applied to the tissues and approximately takes the shape of the stapler jaw. If the material wraps around the jaw, then the grouping of staples tears the material off the stapler jaw.
7) In the case where the material is water absorbing (e.g. a hydrogel or a hydrophilic material), the material can further expand to fill the gaps in the Bs of the staples. A pressure is further created between the Bs of the staples in combination with the material and this pressure further seals the tissue together.
   Bioactive agents, such as pharmaceutical, radionucleotides, nanoparticles, metallic particles, and organic materials can further be placed in the material. The agents can perform functions such as to enhance healing, promote hemostasis, etc.

1. A buttress to reinforce staples released from a surgical stapler comprising:
a. a material adapted for attachment to said surgical stapler wherein the material comprises two states, the first state characterized by a low profile and the second state characterized by a substantially thicker or fuller profile;
wherein said material changes to said second state after said staples are released from the surgical stapler and the surgical stapler released from a tissue.

2. The buttress of claim 1 wherein said material is a hydrogel.

3. The buttress of claim 1 wherein said material is cut into a shape by the mechanism of the surgical stapler traversing said material.

4. The buttress of claim 1 wherein said material releases a bioactive material.

5. The buttress of claim 1 wherein said material is a biodegradeable material.

6. The buttress of claim 1 wherein said second state is related to said first state by the degree of water content.

7. The buttress of claim 1 wherein said second state is related to the first state by the degree of cross-linking of the material.

8. The buttress of claim 1 wherein said material is an elastomer.

9. The buttress of claim 1 wherein said material is inflateable with a gas or fluid.

10. The buttress of claim 1 wherein said material is a container adapted to hold a fluid.

11. The buttress of claim 1 wherein said material is a crosslinked polymer.

12. The buttress of claim 1 wherein said material is activated after application to the tissue.

13. The buttress of claim 1 wherein the material is transparent.

14. A method of creating a secure staple line comprising:
a. applying a material to at least one jaw of a surgical stapler in a first state;
b. sterilizing the surgical stapler and the material;
c. applying the surgical stapler and material to the tissue of a subject;
d. allowing the material to change state to a second state after application of the stapler to the tissue.

15. The method of claim 18 further comprising applying an energy to said material to further secure the staple line.

16. The method of claim 14 further comprising releasing a bioactive material from said material.

17. The method of claim 14 wherein said material is a hydrogel.

18. The method of claim 14 wherein said material is an elastomer.
